(19) United States
(12) Patent Application Publication
(10) Pub. No.: US 2026/0199655 A1
(43) Pub. Date: Jul. 16, 2026

(54) Sleeve for a Medical Tube Connection

(71) Applicant: Xenios AG, Heilbronn (DE)

(72) Inventors: Markus Hügel, Thalheim (DE); Lotte Schraven, Aachen (DE); Georg Wagner, Aachen (DE)

(21) Appl. No.: 19/135,291

(22) PCT Filed: Dec. 13, 2023
(86) PCT No.: PCT/EP2023/085703

§ 371 Date: Jun. 3, 2025

(30) Foreign Application Priority Data
Dec. 14, 2022 (DE) 10 2022 004 696.2

(51) Int. Cl. A61M 39/12; A61M 39/10

(52) U.S. Cl. CPC A61M 39/12; A61M 39/1011

(57) ABSTRACT

The present disclosure relates to sleeves for providing a medical tube connection, e.g., to provide a fluid-tight and mechanically stable connection between a medical tube and a connector received therein. Accordingly, a sleeve is suggested comprising a first sleeve part and a second sleeve part that are connectable to each other, wherein each sleeve part comprises two side walls which are connected to each other and form an inner wall defining an inner cavity and having a continuous semi-circular section in cross-section. The inner walls are adapted to circumferentially surround a medical tube portion received in the inner cavity in a connected state of the sleeve parts. Each semi-circular section comprises a sealing portion and a fixation portion which are formed uninterrupted, continuous and parallel to each other in a circumferential direction.

### CROSS-REFERENCE TO RELATED APPLICATIONS

The present application is the national stage entry of International Application No. PCT/EP2023/085703, filed on Dec. 13, 2023, and claims priority to Application No. DE102022004696.2, filed in the Federal Republic of Germany on Dec. 14, 2022, the disclosures of which are expressly incorporated herein in their entirety by reference thereto.

### TECHNICAL FIELD

The present disclosure relates to sleeves for providing a medical tube connection, e.g., to provide a fluid-tight and mechanically stable connection between a medical tube and a connector received therein.

### BACKGROUND

For medical devices or systems which require a tube or hose or tube set when in use, such as a dialysis system and an extracorporeal circulatory support system, it is important to ensure that the respective hose or tube is connected in a fluid-tight manner and is adequately secured. It is therefore important to prevent a fluid from leaking due to a faulty sealing and/or an unintentionally dislodged tube connection. For example, for a therapeutic treatment of a patient, one or more tubes may be provided for conveying a medical fluid or blood of the patient. The respective tube is connected to a corresponding medical device, for example a pump or oxygenator, or to another tubing or attachment by means of a tube connector. Such a connector is usually provided with a conical portion being radially tapered in a longitudinal direction, which is accordingly inserted into the tube at one end thereof and is received therein. The connector and, in particular, the tapered portion, are typically sized for an internal diameter of the tube.

To prevent leakage from occurring or developing between the tube and the conical portion of the connector, also during long-term use, a fixing ring is slid over the tube according to conventional methods. The fixing ring is to be positioned such that it presses the wall of the tube against the conical portion of the accommodated connector at a designated point, thereby providing an adequate fluid sealing. Furthermore, the fixing ring should counteract the occurrence of dead spaces between the connector and the tube over the course or duration of therapy, which should be avoided from a therapeutic point of view. After correct positioning, an adhesive is applied to fix the fixing ring to the tube, for example, a UV adhesive, which fixes the fixing ring in its position after the curing process.

Furthermore, according to the prior art, a cable tie is attached to the tube for strain relief. The cable tie is typically positioned and tightened with an axial offset to the fixing ring and behind an edge, for example an end edge or a barb edge of the conical portion. By means of such fixation the correct position of the fixing ring relative to the connector and thus its correct function is also ensured.

However, the attachment and positioning of the fixing ring and the cable tie is performed manually and successively, such that the provision of a secure tube connection in this manner is error-prone and time-consuming. Furthermore, there is a risk of low reproducibility due to the required process steps.

### SUMMARY OF THE INVENTION

It is advantageous to improve the provision of medical tube connections and, in particular, to realize a simplified tube connection with improved reproducibility.

According to the present disclosure, it has been recognized that in particular the assembly of the fixing ring requires an exact positioning in order to be able to provide the desired tightness. In some implementations, the positioning should be controlled by force-displacement in order to be able to compensate for any present manufacturing tolerances of all components. If the fixing ring is not positioned exactly, leakage and/or dead spaces can occur. In case of a blood tube, dead spaces can lead to thrombi that are dangerous for the patient. Larger leaks can also endanger the patient and furthermore pose a hygiene problem. Furthermore, the application of the fixing ring requires the application of an adhesive, wherein the fixing ring must be held securely in position during the curing process to prevent accidental slippage.

It has also been recognized that manual positioning and pinpoint or exact alignment of the cable tie are also prone to error. This is because the cable tie is advantageously not fastened immediately adjacent to the barb edge, in order to reduce excessive tensile stress on the tube wall and accordingly not to stress the tube wall too much. At the same time, the distance to the barb edge should not be too large in order to ensure strain relief and correct positioning of the fixing ring. Furthermore, it was found that the tightening force of the cable tie is accompanied by a low reproducibility.

Based on the known prior art, it is therefore an advantage of embodiments of the present methods, systems, and devices to establish an improved tube connection and, e.g., a simplified tube connection with improved reproducibility.

Accordingly, a sleeve for providing a medical tube connection is suggested, comprising a first sleeve part and a second sleeve part which are connectable to each other. Each sleeve part comprises two side walls which are connected to each other and form an inner wall defining an inner cavity having a semi-circular section which is continuous in cross-section. The inner walls are adapted to circumferentially surround a medical tube portion received in the inner cavity when the sleeve parts are connected. Each semi-circular section comprises a sealing portion and a fixation portion, which are formed uninterrupted, continuous and parallel to each other in a circumferential direction.

According to the present disclosure, the functions of a fixing ring and a cable tie can be combined by a single sleeve by means of the corresponding sealing portion and fixation portion, respectively. The fixation portion ensures that a hose or tube received in the inner cavity is securely held relative to a connector received in the tube, at least in the longitudinal direction of the tube. Thus, even when tensile forces are applied to the tube, there is no slippage of the sealing provided by means of the sealing portion. Advantageously, the sealing portion is firmly connected to the fixation portion via the inner wall or the semi-circular section.

Thus, on the one hand, it is ensured that due to the arrangement of the sealing portion in axial respect or in longitudinal direction and relative to the fixation portion, which is predefined in the semi-circular section, a precise fitting and best possible positioning of the sealing portion is provided when the sleeve parts are guided around the tube and connected to each other at the level of the connector.

A manual positioning of a separate sealing ring or fixing ring can be effectively avoided in this way, which may significantly improve the reproducibility of the provided seal.

On the other hand, since the fixation portion is already provided in the semi-circular section, a separate fastening or fixing of the sealing portion is no longer necessary. Thus, the application of an adhesive can be dispensed with. An associated curing time and potentially cumbersome maintenance of a position can be avoided. This advantageously simplifies and shortens the assembly or the assembly is considerably simplified. Since the assembly is typically carried out in a clean room or a sterile environment, the available space can also be used more efficiently by dispensing with corresponding assembly workstations.

The configuration of the sealing portion and the fixation portion as uninterrupted, continuous and radially parallel structures further provides a rotational symmetry of the semi-circular section. This allows a tube portion received in the inner cavity to be evenly delimited or biased at its circumference by the sealing portion and the fixation portion. The “parallel arrangement” of the portions is to be understood such that the sealing portion and the fixation portion are arranged successively after each other in the longitudinal direction, such that the tube portion contacts or engages both the sealing portion and the fixation portion at its received circumference in the longitudinal direction. The longitudinal direction furthermore extends perpendicular to the cross-section of the inner cavity, i.e. perpendicular to the lateral extension defined by the side walls, and thus typically corresponds to the longitudinal extension of the tube and the connector received in the tube.

The two-part configuration of the sleeve with a first sleeve part and a second sleeve part further enables the medical tube connection to be simplified. For example, the tube portion may first be inserted into the inner cavity of the first sleeve part. In this way, the circumference of the tube portion is surrounded by the semi-circular section of the inner wall of the first sleeve part and extends longitudinally through the inner cavity. The second sleeve part may then be passed around the same tube portion such that the semi-circular section of the inner wall of the second sleeve part surrounds the section of the tube circumference that is not surrounded by the inner wall of the first sleeve part. The first sleeve part and the second sleeve part are thus connected to jointly form the sleeve and provide the tube connection. The initially unconnected state of the sleeve parts facilitates the placement or insertion of the tube portion to be connected. Thereby, a tube connection can be provided, which does not require any special qualification of the operator or user.

The first sleeve part and the second sleeve part may accordingly together form a substantially annular or ring-shaped sleeve. The semi-circular sections do not have any offset to each other in the longitudinal direction, but merge into each other or overlap in the longitudinal direction when the sleeve is connected. The inner cavities form a single cavity in which the total circumference of the tube section is accommodated. Hence, the overall cavity is defined at least by the semi-circular sections. The inner cavities accordingly form a channel or through-hole or bore, wherein a radius of the semi-circular sections or a diameter of the overall cavity may vary and/or can be sized for an outer diameter of the tubing in the region of the connector received therein. In other words, a varying diameter of the overall cavity may advantageously be (precisely) adapted to or correspond to the dimensioning of the connector and the wall thickness of the tube.

For example, the sleeve may be configured for a medical tube to be fluidically connected to a medical device. The overall cavity is sized for a corresponding predefined wall thickness of the tube and a dimensioning of a connector or port of the medical device which is at least partially received or accommodated by a tube end. The medical device may be, for example, a device of a dialysis system, for example a dialyzer or pump, or also a device of an extracorporeal circulatory support system, for example, a (membrane) oxygenator or pump. The tube is then typically adapted to convey a medical fluid and the blood of a patient. In this manner, the sleeve can in particular provide a secure fluid seal or liquid seal for a blood tube of a medical system.

The side walls of the respective sleeve part can be formed integrally with each other, such that each sleeve part can be formed from an integral part, e.g., manufacturable by means of a cost-efficient injection molding process.

In some embodiments, the semi-circular sections are identically formed for the sleeve parts and/or are arranged without offset to each other in the longitudinal direction in the connected state. In this way, the semi-circular sections may overlap in the longitudinal direction. Furthermore, they can be identically formed. In this way, a level of rotational symmetry of the inner wall around a received or inserted tube portion and a connector portion inserted or received therein may be provided. Thereby, the exact orientation of the sleeve parts in the circumferential direction of the tube portion is irrelevant. This is particularly advantageous for the provision of a tube connection with a rotationally symmetrical connector.

In order to provide a uniform or even seal and uniform or even fixation or attachment to a tube portion accommodated in the sleeve, the inner cavities can together form a substantially circular inner cavity in the connected state and in cross-section and/or the sleeve parts can be formed in such a way that the inner cavity is defined only by the semi-circular sections. Thus, the circumference of the tube portion can be completely enclosed by the semi-circular sections. In this way, it can be avoided that the overall inner cavity is provided by a (substantial) portion of the inner wall which does not form or does not need to form a semi-circular section, such that, for example, an at least partially oval or ellipsoidal shape of the cross-section can be avoided and a uniform or even contact between the sealing portion and the tube circumference as well as between the fixation portion and the tube circumference can be provided.

The first sleeve part and the second sleeve part may be at least partially connected to each other in the unconnected state of the sleeve. This may be provided, for example, by means of a hinge formed or provided on two adjacent side walls of the two sleeve parts or by means of a flexible connecting arm. In some embodiments, however, the first sleeve part and the second sleeve part are unconnected and separate sleeve parts in the unconnected state. This can further facilitate the provision of the medical tube connection and the placement or insertion of the tube portion.

The first sleeve part and the second sleeve part may also be releasably and/or reversibly connectable to each other. Accordingly, a connecting mechanism may be provided, for example by means of at least one latching element, which enables disassembly and/or readjustment after actuation of the connecting mechanism. In this way, an existing tube connection to a medical device may be easily disconnected, for example for successive treatment. Likewise, a contact or clamping pressure of the sealing portion and the fixation portion may be adjusted to a received tube portion such that a desired or required seal or fixation is provided.

To provide an improved seal of a tube portion to a connector received therein, the sealing portion of a respective sleeve part preferably defines an inner surface which is inclined towards the inner cavity.

The inner surface is thus inclined to the longitudinal axis of the sleeve. An angle of inclination of the inner surface of the sealing area is preferably predefined and corresponds to an angle of inclination of a connector portion inserted in the tube portion. The inner surface of the sealing portion can thus run parallel to the connector portion. This achieves a planar contact to and thus a planar or surface pressing of the tube portion.

In some embodiments, the inner surface of the sealing portion may be formed to provide a contact surface with the received medical tube in the connected state of the sleeve and in the received state of a medical tube.

The inclined contact surface, which can be parallel to the connector portion, may thus provide a significant improvement in terms of sealing and substantially prevents a point contact. Due to the contact surface and, e.g., a parallel guidance or extension, the inclined contact surface in the connected state causes the tube wall to engage evenly with the surface of the connector inserted in the tube portion. Thus, dead spaces in the interior of the tube can be advantageously avoided and, at the same time, an improved pressure distribution can be provided with regard to the tube wall.

However, the angle of inclination of the inner surface may also deviate at least in sections from the angle of inclination of the connector portion. This may provide a line contact, with the greatest compression occurring along a circumferential line. A sealing may thus be improved for a predefined section of the connector. In some embodiments, the angular difference may provide self-locking between the conical elements of the connection.

In some embodiments, the sealing portion is tapered in the longitudinal section of the sleeve. In this way, the inner surface of the sealing portion can be linear or stepless in a longitudinal section. Its radial extension, starting from the inner wall, can essentially change at a predefined angle of inclination. In particular, the radial extension of the inner surface of the sealing portion may increase progressively from a longitudinal end portion closest to the fixation portion to the opposing longitudinal end portion of the inner surface of the sealing portion.

The conical configuration has the advantage that a uniform contact pressure can be provided from the tube wall to a connector received therein. The conical configuration or design of the inner surface of the sealing portion corresponds in particular to a conical configuration of the corresponding connector portion.

In order to provide a uniform fixation and the largest possible contact area between the fixation portion and the circumference of a received tube portion, the fixation portion can define an inner surface with a constant radius. Such an inner surface can extend essentially parallel to the inner wall or semi-circular section, but can be spaced therefrom by a constant radius.

In this manner, a contact surface can also be provided, which is parallel to a substantially cylindrical portion of a connector portion received in the tube portion. Such a cylindrical portion is typically provided behind a barb edge of a conical portion of a connector. Thus, in the connected state of the sleeve and in the received state of the tube portion, a uniform contact force can be provided to secure the tube wall to the connector. Due to the contact surface, a provided contact force or pressure can be increased and a potentially less advantageous stress on the tube wall can be reduced.

Alternatively, the inner surface of the fixation portion may also be formed at least partially convex towards the inner cavity. Such configuration has the advantage that a tube wall is not excessively stressed even at a high contact pressure and/or no edges exist between an end edge or barb edge of the connector and the inner surface of the fixation portion. Even if the tube wall is clamped longitudinally by the fixation portion when the sleeve parts are connected, kinking of the tube wall can be avoided to the largest possible extent.

In some embodiments, at least at a longitudinal end portion towards the sealing portion, the fixation portion comprises a wall which extends substantially perpendicular to the inner wall and/or exclusively radially into the cavity. As described above, common tube connectors typically comprise a conical portion and a cylindrical portion adjacent a corresponding barb edge. Thus, the provision of a substantially vertical or radial wall in the fixation portion not only has the advantage of enabling a spacing of a contact surface of the fixation portion towards the inner wall, which has an advantageous effect on the contact force. This approach also has the effect of providing a contact surface with a substantially perpendicular adjacent connector portion at the barb edge. In this manner, an improved fixation of the tube wall to the connector portion can be provided, wherein the perpendicular wall of the fixation portion may further form a stop element or retaining element. This may prevent the fixation portion from being inadvertently pulled over the barb edge when a pulling force is applied.

In some embodiments, the vertical wall is adjacent to an inner surface of the fixation portion with a constant radius or with a convex section. In this way, starting from a longitudinal end closest to the sealing portion, the fixation portion may initially be vertical and then parallel to the inner wall or curved towards the inner cavity.

To avoid potentially less desirable linear contacts or contact points, the wall can be rounded at the radial end opposite the inner wall such that the wall and inner surfaces adjacent thereto have no sharp edges.

To accommodate an end edge or barb edge of a connector received in the tube portion, the sealing portion and the fixation portion can be longitudinally spaced apart by a gap. The sealing portion and the fixation portion thus extend at least partially radially from the inner wall into the inner cavity. The maximum radial extension may be different or substantially the same depending on the configuration or design of the connector.

However, between the sealing portion and the fixation portion, the radial extension of the inner wall is smaller. The radius of this gap or recess may be adapted to the end edge of the connector. For example, the longitudinal spacing and/or radial depth of the gap may be sized for a predefined tapered portion and/or radial extension of an end edge of a connector. In this way, the sealing portion overlaps longitudinally with a predefined portion of the connector in the connected state of the sleeve and in the received state of a tube portion.

According to the present disclosure, a predefined seal can be provided at a conical portion of the connector when the sleeve is connected, in order to avoid the occurrence of leaks and/or provision of dead spaces as far as possible. This also has the advantage that the sealing portion and the fixation portion may be biased into the intended position upon connecting the sleeve. This further simplifies the positioning of the sleeve.

In some embodiments, the sealing portion and the fixation portion are configured to engage a connector inserted in the tube in a form-fitting manner in a connected state of the sleeve and in a received state of a medical tube.

For example, the sealing portion and the fixation portion may be configured to receive an end edge of the connector between the portions in the connected state of the sleeve to prevent a longitudinal displacement of the sleeve relative to the connector. As explained above, a gap may be provided for this purpose. Alternatively, or additionally, the sealing portion may, for example, also have an inclined contact surface or be formed conical overall. In this way, the sealing portion is delimited by a corresponding conical portion of the connector, at least in one longitudinal direction. A movement of the sleeve in an opposite longitudinal direction may be prevented, for example, by means of a vertical wall of the fixation portion, wherein such wall may be delimited by a corresponding end edge of the connector.

In order to facilitate an assembly or a connection of the sleeve parts with a tube portion received therein, the contour of the sleeve, which is substantially defined by the side walls of the sleeve parts, may be at least partially divided between the sleeve parts at adjacent side walls. For example, at least the first sleeve part has a projection, e.g. a nose or lug on at least one side wall at an end in the circumferential direction that is free in the unconnected state. The projection may extend from the inner wall and from said end in the circumferential direction. In some embodiments, at least the inner wall of the second sleeve part has, on at least one side wall, at an end portion which is free in the circumferential direction and in the unconnected state, a radial recess for receiving the projection.

According to the present disclosure, the inner wall of a side wall may have a radial offset at its end portion such that the inner wall defines a larger radius of the inner cavity. The projection of an adjacent inner wall of the other sleeve part can be inserted into the corresponding wall recess. In this way, an interference fit can be facilitated when connecting the sleeve parts and a certain tolerance can be provided for the received tube portion. Due to the recess, expansion of the tube portion can be taken into account when joining the sleeve parts and entrapment of the tube portion between the end portions of the adjacent side walls can be avoided as far as possible. However, the projection ensures that any leakage can be prevented due to an otherwise reduced contact pressure on the tube circumference. Accordingly, the projection is advantageously formed from a flexible material and/or has a thickness that is less than the thickness of the inner wall. This can ensure a certain flexibility and deformation of the projection when connecting the sleeve parts.

The projection and the recess extend at least partially in the longitudinal direction. In some embodiments, a projection is provided at both corresponding end portions of the side walls of the first sleeve part and a recess is provided at both corresponding end portions of the side walls of the second sleeve part. In this way, the relative alignment of the sleeve parts to each other can be simplified when connecting the sleeve parts.

Alternatively, however, it can also be provided that each sleeve part has a corresponding end portion of a side wall provided with a corresponding projection, wherein the radially opposite end portion comprises a corresponding recess. In this way, for example, first one side wall of the first sleeve part can be partially connected to one side wall of the second sleeve part and subsequently the other side wall of the first sleeve part can be connected to the other side wall of the second sleeve part. A quasi stepwise connection of the sleeve parts can thus be made possible or facilitated.

In some embodiments, the at least one projection and the at least one recess extend in longitudinal direction exclusively over or along the sealing portion. In this way, leakage in the most relevant area of the connector or the tube portion can be avoided to the largest possible extent. At the same time, a relative alignment of the sleeve parts required for connecting the sleeve parts is thereby indicated. The connection of the sleeve parts can thus be simplified and the user can be further supported in a cognitively simple manner. The limitation to the sealing portion furthermore has the effect that a predefined contact pressure at the level of the fixation portion is not, or at least not significantly, influenced by the existing projection(s) and recess(es).

The at least one projection can further be beveled in a radial direction. Thereby, the effect on the corresponding portion with regard to the contact pressure force to be provided can be reduced and, for example, be limited to a longitudinal end portion of the sealing portion.

In addition, or alternatively to the above-described configuration of the sleeve by means of at least one projection and at least one recess, at least the first sleeve part may have a projection on at least one side wall at an end which is free in the circumferential direction in the unconnected state. The projection extends from the inner wall and starting from said end in the circumferential direction and is arranged in such a way that, in the connected state, it engages with a longitudinal end of an adjacent side wall of the second sleeve part.

Thus, for example, a projection can be provided on each side wall in front of the inner wall, for example at the longitudinal end portion of the sealing portion, wherein said projection, in the connected state of the sleeve, closes or terminates at the longitudinal end of an adjacent side wall of the respective other sleeve part.

In order to connect the two sleeve parts to each other, the first sleeve part and the second sleeve part can comprise a connecting mechanism which is respectively arranged radially outside of the semi-circular section. The connection of the sleeve parts can be provided in different ways. However, the arrangement of the connecting mechanism radially outside of the semi-circular section allows the sleeve to be completely closed. The semi-circular sections of the sleeve parts adjoin and/or are brought flush or aligned with each other. This allows a substantially circular internal cavity to be formed in the cross-section of the sleeve. Furthermore, this enables a secure guidance of the sleeve parts relative to each other when connecting the sleeve parts. Also, it is largely prevented that a received tube portion is squeezed by the connecting mechanism due to expansion during connection.

Alternatively, it may also be provided that the connecting mechanism extends without radial offset from, for example, the inner wall or the respective side wall. In this way, the inner wall between the semi-circular sections may partially define the inner cavity, such that a fully circular inner cavity may potentially not be provided. However, this provides a level of clearance or tolerance for different outer diameters of the tube portion to be received or accommodated. Likewise, it may reduce the radial or lateral extension of the sleeve parts.

In some embodiments, the connecting mechanism of a sleeve part has at least one latching element at each end portion of the side wall which is free in the circumferential direction and in the unconnected state. Such latching element may extend from the respective end portion. Also, the connecting mechanism of the other sleeve part may have a retaining element at each of the end portions of the side walls that are adjacent in the connected state for holding the respective latching element in a form-fitting manner.

The at least one latching element may, for example, be in the form of a radially extending hook. The retaining element may preferably be formed as a corresponding edge or groove to engage with and receive the hook. The retaining element may, for example, be provided in a pocket or recess in the respective side wall such that the latching element can be inserted into the pocket and engage with the retaining element. In this way, a guide for the latching element and thus a relative alignment of the sleeve parts to each other can also be supported or even predefined. Alternatively, however, the retaining element can also be arranged on an outer surface of the side wall in order to enable a grippable and potentially releasable or adjustable connection in this manner.

The at least one latching element and retaining element can be configured to be connected to each other in a snap-fit manner. For example, it may be provided that the latching element has a chamfer which serves as a guide when sliding along the retaining element in the direction towards the retaining element. However, it can form a stop element in the opposite direction, which restricts or even prevents movement.

The latching element and the retaining element are preferably arranged in such a way that the inner walls of the sleeve parts can be connected to each other or can adjoin each other and are held securely in such an arrangement.

The configuration of the at least one latching element and the corresponding at least one retaining element thus enable a simple assembly or connection of the sleeve parts with further improved reproducibility.

In some embodiments, the connecting mechanism of the one sleeve part has a plurality of latching elements at each free end portion. These may be spaced apart to each other at a predefined distance along a substantially linear extension from the respective end portion. Depending on the configuration or design of the sleeve parts, the linear extension can be understood as a perpendicular extension to an end face of the side wall which, in the connected state of the sleeve, is adjacent to a corresponding end face of the side wall of the other sleeve part. The linear extension may, alternatively or additionally, be co-linear with the extension of the end portion of the side wall and/or tangential or parallel to the end portion of the inner wall. In simplified terms, the latching elements may preferably be connected to each other. In some embodiments, at the level of the end of the semi-circular section, they may extend substantially straight to the adjacent end of the semi-circular section of the other sleeve part.

The provision of multiple latching elements enables a tolerance compensation for deviations in the predetermined outer diameter of the tube portion to be accommodated. In this way, a contact pressure can be varied and set lower in the case of oversizing and higher in the case of undersizing. In this way, the tube portion can be adequately sealed and held due to the corresponding latching element. In some embodiments, the latching elements are spaced at the same distance from each other, such that a fine adjustment of the contact pressure and the connection of the sleeve parts can be facilitated by successive adjustment.

In another possible embodiment of the sleeve, both sleeve parts are identical. This can further simplify the assembly. In the case of a plurality of identical sleeve parts, each sleeve part can be formed to match a respective other sleeve part.

### BRIEF DESCRIPTION OF THE DRAWINGS

Further embodiments of the present disclosure will be explained in more detail by the following description of the Figures.

FIG. 1 shows a schematic perspective view of a sleeve according to the present disclosure;

FIG. 2 shows a detailed view of the semi-circular section according to FIG. 1 in a longitudinal section;

FIG. 3 shows a semi-circular section according to the present disclosure in engagement with a tube portion and a connector portion accommodated therein;

FIG. 4 shows a schematic longitudinal section of the semi-circular sections of the first sleeve part and the second sleeve part in the connected state of the sleeve and with a tube portion received therein;

FIG. 5 schematically shows a connecting mechanism of the sleeve parts;

FIGS. 6 and 7 show, in perspective and with a detailed view, an embodiment wherein the contour of the sleeve parts is divided by means of projections and recesses;

FIGS. 8 and 9 show a sleeve part according to FIG. 6 with alternative designs of the projections; and

FIG. 10 shows a schematic side view of alternative arrangements and configurations of the projection(s) and recess(es).

### DETAILED DESCRIPTION

In the following, embodiments will be explained in more detail with reference to the accompanying Figures. In the Figures, corresponding, similar, or like elements are denoted by identical reference numerals and repeated description thereof may be at least partially omitted in order to avoid redundancies.

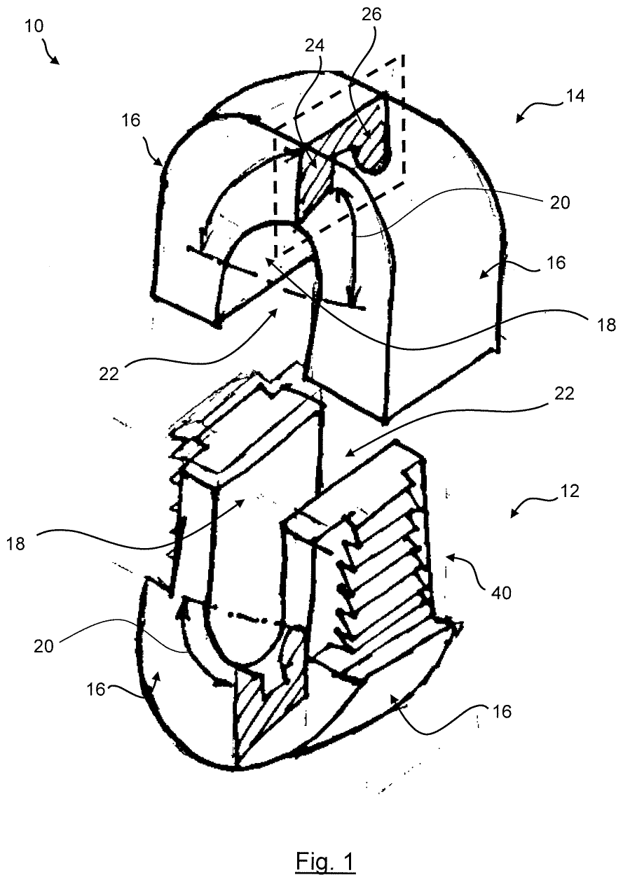

FIG. 1 schematically shows a sleeve 10 according to the disclosure in a perspective view, wherein the sleeve 10 is formed by a first sleeve part 12 and a second sleeve part 14, which are shown in FIG. 1 in an unconnected state. The sleeve 10 is formed to provide a medical tube or tubing connection, wherein the sleeve 10 provides a permanent fluid-tight connection between a medical tube or corresponding tube portion and a connector portion inserted in the tube portion. For this purpose, the sleeve parts 12, 14, which are connectable to one another, each comprise two side walls 16, which are integrally formed from a single part in the present example. The side walls 16 extend laterally and in the circumferential direction and, in the unconnected state of the sleeve 10, each have only one free end in the circumferential direction. Together, the side walls 16 form an uninterrupted or continuous inner wall 18 which comprises a semi-circular section 20, which is continuous in cross-section, as shown by the dashed lines and arrows in FIG. 1. In this manner, the inner wall 18 defines an inner cavity 22 which is delimited radially outwards by the inner wall 18 and in particular by its semi-circular section 20.

The inner cavity 22 further extends in a longitudinal direction and thus forms a receptacle for a tube portion (not shown). In this way, the tube portion can be inserted at least into the semi-circular cross-section of the inner cavity 22 and be enclosed by the inner wall 18. Thus, when the sleeve parts 12, 14 are connected, the circumference of a corresponding medical tube portion can be fully encompassed by and engaged with the inner wall 18. The semi-circular sections 20 each have a sealing portion 24 and a fixation portion 26 which are circumferentially uninterrupted, continuous and parallel to each other. In other words, the semi-circular sections 20 may be ring-shaped. The sealing portion 24 and the fixation portion 26 have longitudinally different ring portions with corresponding circumferentially constant geometries. In this way, a single sleeve 10 can fulfil two functions in the connected state: namely, on the one hand, a seal towards the connector and, on the other hand, a fixation of the received tube portion.

A connection of the sleeve parts 12, 14 can be enabled by a connecting mechanism 40, which is arranged radially outside of the semi-circular section 20.

The semi-circular section 20 extends along the inner wall 18 and may be provided along or form the entire inner wall 18. Alternatively, the inner wall 18 may extend beyond the semi-circular section 20, for example, tangentially or perpendicularly to an end portion or end face of the side wall 16.

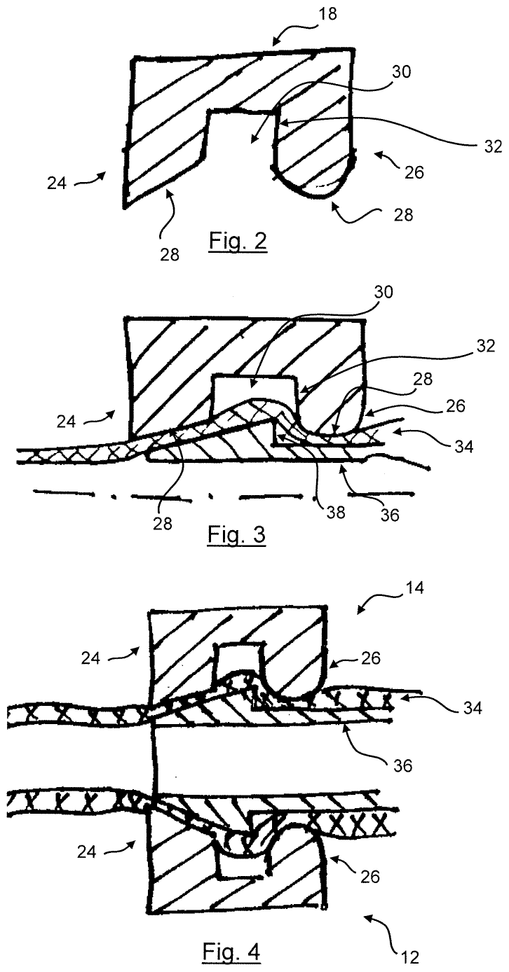

FIG. 2 shows the semi-circular section 20 of FIG. 1 in a detailed view, as shown by the dashed section in FIG. 1, which corresponds to a longitudinal section. Accordingly, the sealing portion 24 comprises an inner surface 28 which is inclined towards the inner cavity 22. Its radial extension increases continuously from the inner wall 18 towards the inner cavity 22 and relative to the side wall 16 or its outer wall. Accordingly, the sealing portion 24 in the present example is conical in shape, wherein the inner surface 28 extends to the further inner wall 18 or to the side wall 16 with a predefined angle of inclination. The inclined inner surface 28 of the sealing portion 24 is advantageously formed as a contact surface and is oriented in such a way that it corresponds to an angle of inclination of a connector portion 36 inserted in the tube portion 34, as shown in FIG. 3.

When the sleeve parts 12, 14 are connected to each other and the tube portion 34 is inserted in the inner cavity 22 such that it extends longitudinally through the sleeve 10, the inner surface 28 of the sealing area 24 engages the hose or tubing wall and the tapered section of the connector portion 36. Due to the angle of inclination, this enables a gapless seal of the tube portion 34 towards the connector portion 36 such that no (potentially hazardous) dead spaces are present or created.

Furthermore, the fixation portion 26 comprises an inner surface 28 which is curved and convex towards the inner cavity 22. As a result, in the connected state of the sleeve 10 and in the inserted state of the tube portion 34, a potentially disadvantageous pinching of the tube wall or a double fit towards a barb edge or end edge 38 can be avoided. Furthermore, due to the shape design, biasing of the sleeve 10 into a predefined position relative to the connector portion 36 is enabled, which facilitates assembly of the sleeve to provide the tube connection.

In the present example, the curved or convex portion or inner surface 28 of the fixation portion 26 extends from a substantially vertical wall 32 which abuts or defines a longitudinal extension of a gap 30 between the sealing portion 24 and the fixation portion 26. The fixation portion 26 and the sealing portion 24 are longitudinally spaced apart by the gap 30. The substantially vertical wall 32 of the fixation portion 26 allows the sleeve 10 to be retained relative to the tube portion 34 and the connector portion 36 in a form-fitting manner when connected. Movement is restricted longitudinally in one direction, on the one hand, by the end edge 38 and the wall 32, and in the other direction, on the other hand, by the inclined contact surface of the sealing portion 24 and the tapered portion of the connector portion 36. This configuration is illustrated in the connected state of the sleeve 10 with respect to both sleeve parts 12, 14 in FIG. 4. Accordingly, the end edge 38 is received in the gap 30. The connector is thus held in the longitudinal direction by the geometry of the sealing portion and the fixation portion by means of positive locking, i.e. in a form-fitting manner. The sleeve 10 is held in a form-fitting manner relative to the tube portion 34 and the connector portion 36 due to the geometry of the connector portion 36.

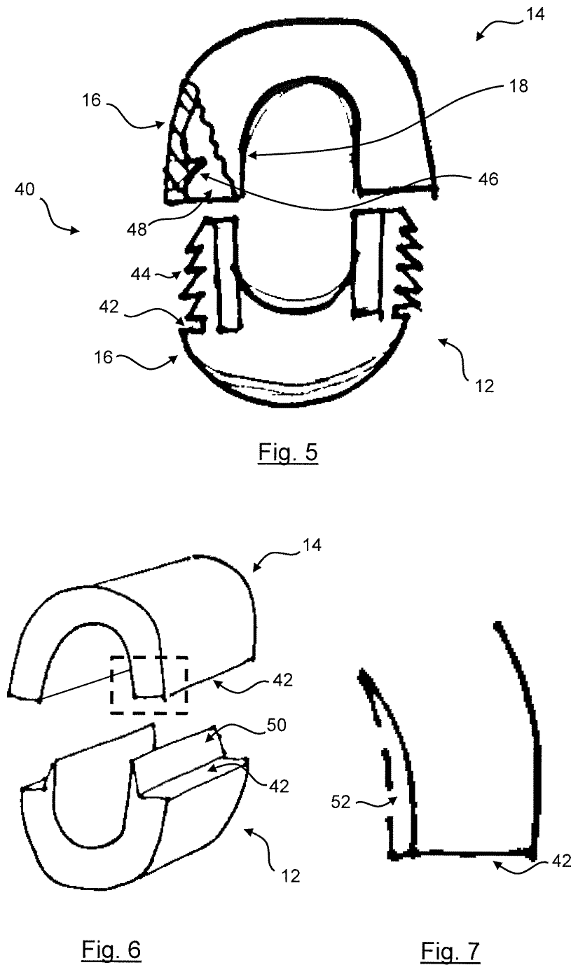

FIG. 5 schematically illustrates an exemplary connecting mechanism 40 of the sleeve parts 12, 14. Both the first sleeve part 12 and the second sleeve part 14 comprise a connecting mechanism 40 which is respectively arranged radially outside of the semi-circular section 20. According to the embodiment shown, the first sleeve part 12 is exemplarily provided with a plurality of latching elements 44 on the respective side walls 16 or their free end portions 42. The latching elements 44 are formed as hooks and are connected to each other. They may extend linearly as a whole and with equal spacing from each other from the respective end portion. In this way, the latching elements 44 extend substantially perpendicularly to a free end face of the side wall 16 when the sleeve 10 is in the unconnected state, but which is overlapped by a corresponding free end face of the adjacent side wall 16 of the other sleeve part when the sleeve 10 is in the connected state.

The connecting mechanism 40 of the second sleeve part 14 comprises, at the end portions 42 of the side walls 16 which are adjacent in the connected state of the sleeve 10, a respective retaining element 46 for holding the respective latching element 44 in a form-fitting manner. The retaining element 46 may be formed as a corresponding edge or groove to engage with the latching hook and receive it in a form-fitting or snap-fitting manner. For this purpose, the latching hook comprises a chamfer which is biased when it slides along and engages the edge. The edge or groove is provided in a pocket 48 or recess so that the latching element 44 can be inserted into the pocket 48 and be brought into engagement with the retaining element 46. The pocket 48 acts as a guide for the latching element 44 and thus defines a relative alignment of the sleeve parts 12, 14 with respect to each other.

An example of how the contour of the sleeve parts 12, 14 may be divided is shown in FIGS. 6 and 7 in a perspective view and a detailed view, respectively. Accordingly, for example, the first sleeve part 12 may have a lug or projection 50 at both free end portions 42 of the side walls 16, wherein said projection 50 extends, for example, tangentially to the inner wall 18, starting from the circumferential end of the inner wall 18. The projection 50 can extend along the entire length of the side wall 16 or its inner wall 18 and thus be provided both in the sealing portion 24 and in the fixation portion 26.

As shown in FIG. 7 with respect to FIG. 6, the inner wall 18 of the second sleeve part 14 comprises a radial recess 52 for receiving a corresponding projection 50 on both side walls 16 and at an end portion 42 which is free in the circumferential direction and in the unconnected state. The inner wall 18 thus has a radial offset at its end portion 42, such that the inner wall 18 defines a larger radius of the inner cavity 22. The nose or projection 50 of an adjacent inner wall 18 of the first sleeve part 12 can be inserted into the corresponding wall recess 52 when the sleeve parts 12, 14 are connected.

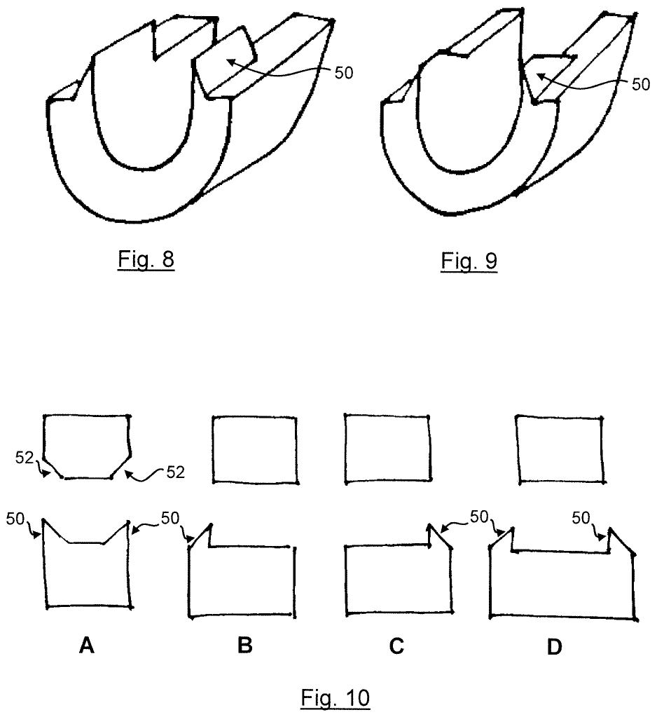

FIGS. 8 and 9 show alternative embodiments of the projections 50 for a sleeve part (for example, the first sleeve part 12). Accordingly, the projections 50 extend longitudinally only over or along the sealing portion 24 of the sleeve 10. The projection 50 shown in FIG. 9 is also radially beveled to allow for an even more compact design and optimized sealing while reducing the risk of leakage.

FIG. 10 shows a schematic side view of alternative arrangements and configurations of the projection(s) 50 and recess(es) 52. Accordingly, FIG. 10A shows one embodiment. At the longitudinal end portions of the sleeve parts there are respectively a projection 50 and a recess 52, such that the contour of the sleeve 10 is divided both in the sealing portion 24 and in the fixation portion 26, but not over their complete extension in the longitudinal direction.

FIGS. 10B to 10D show alternative embodiments of the projection(s) 50, which are arranged at a longitudinal end portion. No separate recess 52 are provided, but the projection 50 is adjacent to the respective longitudinal end face of the side wall 16 of the adjacent sleeve part. Thus, according to FIG. 10B, a projection 50 is provided longitudinally in front of the corresponding sealing portion 24 and according to FIG. 10C, a projection 50 is provided longitudinally behind the corresponding fixation portion 26. Furthermore, according to FIG. 10D, a respective projection 50 is provided at both longitudinal end portions, i.e. both in front of the sealing portion 24 and behind the fixation portion 26. This Figure also shows that the projections 50 can be arranged or dimensioned in such a way that they do not disappear in the overall contour. Thus, they may also sit in front of the contour, as illustrated by the larger dimensioning of the lower sleeve part.

Where applicable, all the individual features depicted in the exemplary embodiments may be combined and/or exchanged without leaving the scope of the invention.

### LIST OF REFERENCE NUMERALS

10 Sleeve
12 First sleeve part
14 Second sleeve part
16 Side wall
18 Inner wall
20 Semi-circular section
22 Cavity
24 Sealing portion
26 Fixation portion
28 Inner surface
30 Gap
32 Wall
34 Tube portion
36 Connector portion
38 End edge
40 Connecting mechanism
42 End portion
44 Latching element
46 Retaining element
48 Pocket
50 Projection
52 Recess

1-19. (canceled)

20. A sleeve for providing a medical tube connection, the sleeve comprising:
a first sleeve part and a second sleeve part that are connectable to each other, wherein each sleeve part comprises two side walls which are connected to each other and form an inner wall defining an inner cavity and having a continuous semi-circular section in cross-section, wherein the inner walls are adapted to circumferentially surround a medical tube portion received in the inner cavity in a connected state of the first sleeve part and the second sleeve part, wherein each semi-circular section comprises a sealing portion and a fixation portion which are formed uninterrupted, continuous, and parallel to each other in a circumferential direction.

21. The sleeve according to claim 20, wherein the semi-circular sections are identically formed for the sleeve parts and/or are arranged without offset to each other in a longitudinal direction in the connected state.

23. The sleeve according to claim 20, wherein the inner cavities together form a substantially circular third inner cavity in cross-section and in the connected state.

24. The sleeve according to claim 20, wherein the first sleeve part and the second sleeve part are formed in such a way that the substantially circular third inner cavity is defined only by the semi-circular sections.

25. The sleeve according to claim 20, wherein the first sleeve part and the second sleeve part are unconnected and are separate sleeve parts in the unconnected state.

26. The sleeve according to claim 20, wherein the first sleeve part and the second sleeve part are releasably and/or reversibly connectable to each other.

27. The sleeve according to claim 20, wherein the sealing portion of each of the sleeve parts defines an inner surface which is inclined towards the inner cavity of the respective sleeve part.

28. The sleeve according to claim 27, wherein the inner surface of the sealing portion is formed to provide a contact surface with the received medical tube in a connected state of the sleeve and in a received state of the tube.

29. The sleeve according to claim 27, wherein the sealing portion is conical in a longitudinal section of the sleeve.

30. The sleeve according to claim 20, wherein the fixation portion defines an inner surface having a constant radius or is formed at least partially convex towards the inner cavity.

31. The sleeve according to claim 20, wherein the fixation portion comprises, at least at a longitudinal end portion towards the sealing portion, a wall which extends substantially perpendicularly to the inner wall and/or exclusively radially into the inner cavity.

32. The sleeve according to claim 20, wherein the sealing portion and the fixation portion are spaced apart from each other in a longitudinal direction by a gap.

33. The sleeve according to claim 20, wherein the sealing portion and the fixation portion are formed to positively engage a connector inserted into the medical tube portion in the connected state of the sleeve and in the received state of the medical tube portion in a form-fitting manner.

34. The sleeve according to claim 20, wherein at least the first sleeve part has at least one projection on at least one side wall at an end which is free in the circumferential direction and in the unconnected state, wherein the at least one projection extends from the inner wall starting from the end which is free and in the circumferential direction, and wherein at least the inner wall of the second sleeve part comprises at least one radial depression for receiving the at least one projection on at least one side wall at an end portion being free in the circumferential direction in the unconnected state.

35. The sleeve according to claim 34, wherein the at least one projection and the at least one recess extend in the longitudinal direction exclusively over the sealing portion, and/or wherein the at least one projection is beveled in radial direction.

36. The sleeve according to claim 34, wherein at least the first sleeve part has a projection on at least one side wall at an end which is free in the circumferential direction in the unconnected state, wherein the projection extends in the circumferential direction from the inner wall and starting from the longitudinal end and is arranged in such a way that, in the connected state of the sleeve, the projection is in engagement with a longitudinal end of an adjacent side wall of the second sleeve part.

37. The sleeve according to claim 20, wherein the first sleeve part and the second sleeve part each comprise a connecting mechanism for connecting the first sleeve part and the second sleeve part, wherein each connecting mechanism is arranged radially outside of the semi-circular section.

38. The sleeve according to claim 37, wherein the connecting mechanism of the first sleeve part comprises at least one latching element extending from each end portion of the side wall being free in the circumferential direction in the unconnected state, and wherein the connecting mechanism of the second sleeve part comprises a retaining element for holding the at least one latching element in a form-fitting manner at each of the end portions, wherein the side walls are adjacent in the connected state.

39. The sleeve according to claim 38, wherein the connecting mechanism of the first sleeve part comprises a plurality of latching elements at each free end portion, wherein the plurality of latching elements are spaced apart from each other by a predefined distance along a substantially linear extension from each end portion.
